(19) United States
(12) Patent Application Publication
(10) Pub. No.: US 2024/0307467 A1
(43) Pub. Date: Sep. 19, 2024

(54) MATRIX FORMULATED WITH PROBIOTICS FOR USE IN THE TREATMENT OF CARIES

(71) Applicants: Diego Alejandro CASTORENA VALENZUELA, Tlaquepaque, Jalisco (MX); Maria Fernanda DE LA ROSA FLORES, Tlaquepaque, Jalisco (MX); INSTITUTO TECNOLÓGICO Y DE ESTUDIOS SUPERIORES DE OCCIDENTE, A.C.

(72) Inventors: Diego Alejandro CASTORENA VALENZUELA, Tlaquepaque, Jalisco (MX); Maria Fernanda DE LA ROSA FLORES, Tlaquepaque, Jalisco (MX)

(73) Assignee: INSTITUTO TECNOLÓGICO Y DE ESTUDIOS SUPERIORES DE OCCIDENTE, A.C., Tlaquepaque, Jalisco (MX)

(21) Appl. No.: 18/274,950

(22) PCT Filed: Oct. 28, 2021
(86) PCT No.: PCT/MX2021/050069

§ 371 Date: Jul. 28, 2023

(30) Foreign Application Priority Data
Jan. 29, 2021 (MX) MX/A/2021/001226

(51) Int. Cl. A61K 35/747; A61K 47/26; A61K 47/36; A61P 1/02

(52) U.S. Cl. CPC A61K 35/747; A61K 47/26; A61K 47/36; A61P 1/02

(57) ABSTRACT

A matrix formulated with probiotics, which can be used to reduce the development of caries by inhibiting Streptococcus mutans and wherein the probiotics include: 3-5 g/100 mL pectin; 6-9 g/100 mL dextrose; 25-50 mg/100 mL citric acid; 2-4 mL/100 mL natural flavouring; and 4×10 12 CFU/mL of the microorganisms Streptococcus salivarius and Lactobacillus plantarum in active state. The matrix is administered to a patient to prevent dental caries.

### FIELD OF THE INVENTION

The present invention is related to biotechnology and microbiology in general, and in particular, it is related to compositions for preventing and treating diseases associated with oral health. More specifically, it refers to a matrix formulated with probiotics to provide stability to beneficial chemical-microbial properties in oral health.

### BACKGROUND OF THE INVENTION

Caries and periodontitis are caused by an imbalance in the bacterial populations of biofilms that are naturally formed and that help maintain the normal state of the oral cavity. A healthy biofilm can be made up of more than 700 bacteria, of which less than 1% are potentially pathogenic such as Streptococcus mutans, which in 95% of cases is the main cause of caries formation. The dental surface is an essential natural habitat for this bacterium, and the tropism for dental biofilm is reflected in its adaptation to synthesize glucans, to bind compounds and to adjust its aciduricity.

Glycosyltransferases (Gtfs) from Streptococcus mutans are adsorbed to produce glucans in situ on the enamel, providing sites for avid colonization, and they adhere firmly through developing peptides. This makes simple removal impossible, requiring a visit to a dentist.

Currently, dentistry is the only health science that combats these diseases in critical states through techniques such as extraction and restoration using endodontics or crowns. On the other hand, biotechnology applied to dentistry is an innovative and minimally invasive alternative. Species such as Streptococcus salivarius and Lactobacillus plantarum are considered Generally Recognized As Safe (GRAS) by the Food and Drug Administration, commonly known by its English acronym FDA; when these species are used as probiotic, they locally inhibit bacterial growth and adhesion, modulating the immune response through the production of antiviral agents.

The present invention refers to a matrix formulated to provide stability to beneficial chemical-microbial properties in oral health.

A search was carried out to determine the closest state of the art, finding the following document. Patent application document published as WO2010077795A3 by Roughead Zamzam Kabiry and Kaspar Kala Marie dated Dec. 14, 2009 was found. This document discloses compositions and methods for preventing and treating diseases associated with oral health. The compositions and methods can improve oral health by reducing the incidence of aspiration pneumonia, community-acquired pneumonia, and nosocomial pneumonia, allowing less gingivitis and plaque, and providing improved tongue flora. By using probiotics naturally present in the oral cavity or gastrointestinal tract, the compositions and methods can reduce anaerobic gram-negative bacilli and increase the presence of normal flora.

Said document discloses a composition for improving oral health comprising a therapeutically effective amount of beneficial bacteria wherein said beneficial bacteria comprise at least one bacterium normally selected from the group consisting of: i) Streptococcus Salivarius; ii) Lactobacillus reuteri; Lactobacillus plantarum 299 or 299v; iv) Streptococcus salivarius KI 2; v) Lactobacillus reuteri ATCC55730; vi) Lactobacillus johnsonii Lal; vii) Lactobacillus rhamnosus GG; viii) Streptococcus thermophilus NCC 1561; ix) Lactococcus lactis NCC2211 (Pelargon strain); and x) Lacteol.

The composition is in a form such as liquid, solid, semi-solid, or a combination thereof. The composition may be a complete oral nutritional supplement. The composition may also be in a form such as tablets, lollipops, sachets, soluble films, or a combination thereof. The document also discloses that the bacteria are inactive.

The oral microbiota of the gastrointestinal tract (GIT) is made up of more than 500 bacteria that collaborate in the digestion and detoxification of food, in addition to stimulating the immune system. This flora is regulated by peristalsis, the secretion of gastric acid and bile salts; in addition, it generates a higher bacterial contraction gradient in the colon and this can be affected by the food eaten and external factors such as pollution, sunlight, etc. Therefore, the use of probiotics that reach the intestinal microflora is important, since the mucosa of said organ helps regulate symbiotic interaction and strengthens the immune system, reducing diseases such as pneumonia or dysphagia; diseases with a patent application published as WO2010077795A3 that claims their reduction in claims 7, 14, and 17. However, for a probiotic to reach the gastrointestinal system safely and functionally, it must be in an inactive formulation, or commonly known as “lyophilized”. The same patent in claim 9 mentions the protection of different microorganisms of claim 7, in said “lyophilized” and/or inactive state. This allows these inactivated bacteria to become active with gastric juices and strengthen the gastrointestinal flora.

The oral microbiota is a totally different environment, due to its pH conditions, oxygenation, and its more than 700 bacteria present, etc. and it is mainly regulated by saliva and the proteins originated from said liquid. Having different conditions, an inactivated bacterium cannot easily inoculate the oral microbiota because it needs to be activated and given the conditions in the mouth, this is not possible, or its action is limited both in the extent of change and in its temporal persistence. However, it is important to strengthen the oral microbiota, since it also contains pathogenic bacteria that could generate a tropism in the microbial population and give rise to diseases such as gingivitis, caries, bad breath, among many others.

The aforementioned document discloses in its claim 1 the microorganisms Lactobacillus plantarum 299 or 299v and Streptococcus salivarius K12. Lactobacillus plantarum is a probiotic strain capable of residing in the human colonic mucosa since it adheres to the intestinal mucosa, where it modulates the composition of the intestinal microflora, helping to maintain adequate colonization of the gastrointestinal tract and improving digestion and metabolism. By colonizing the gastrointestinal tract, the probiotic can form a protective barrier, thus preventing the adhesion of pathogens, protecting against infections, and stimulating the immune system. Streptococcus salivarius K12 is a strain isolated from the throat of a New Zealand child capable of producing antibiotic bacteriocin that helps counteract the growth of S. pyogenes and inhibits the growth of pathogens such as Haemophilus influenzae, S. pneumoniae and Moraxella catarrhalis, all of which are involved in the etiopathogenesis of acute otitis media.

The aforementioned document discloses in its claims 10 to 12 the use of tablets, lollipops, sachets, soluble films and topicals. Our invention (viscous solution) will be the filling of a chewing gum, gummies, toothpaste or any confectionery product that may contain a filling inside, due to the fact that the probiotic bacteria are required to adhere for a long time on the surface of dental pieces to achieve an inhibitory effect on caries and other diseases, since it will be persistent and will form part of the oral cavity biofilm that makes up the oral microbiota. The biofilm that forms keeps bacterial pathogens out of oral tissues, filling the space that pathogens would invade in the absence of the biofilm, while competing with cariogenic bacteria.

Said document WO2010077795A3 discloses in its claims 7, 14 and 17 that its product helps to reduce the incidence of aspiration pneumonia and dysphagia. Our invention focuses on the prevention of caries, tartar reduction, halitosis and yellowing teeth, etc. Dental caries is one of the main oral health problems and its consequences range from inflammation or infection of the pulp tissue to tooth loss.

Naturally, the tooth surface is covered by a biofilm made up of millions of bacterial cells, salivary polymers and food debris, which over time and without proper cleaning begin to turno into plaque, providing an adhesion site for the colonization and growth of pathogenic bacterial species such as Streptococcus mutans.

Streptococcus mutans plays a central role in the etiology of dental caries because it can adhere to the salivary film of enamel and to other plaque bacteria. This happens because they produce weak organic acids as metabolic by-products of fermentable carbohydrates, which cause the local pH to decrease, causing demineralization of the tooth tissue. Normally, the appearance of Streptococcus mutans in tooth cavities is followed by caries after 6-24 months.

The adhesion of this microorganism to the dental surface is due to the interaction that exists between the PAc protein and other saliva proteins that are adsorbed by the teeth enamel. The number of Streptococcus mutans increases with the initiation of dental caries and decreases when the caries is treated, so there is a correlation between this pathological process.

Plaque control can be achieved by mechanical oral hygiene procedures, but in many cases this is not enough. Therefore, the direct addition of active probiotic products according to our invention into the oral cavity helps to reduce the overall rate of new plaque accumulation, reduce or eliminate existing plaque, and inhibit only the growth of pathogenic species.

Document WO2010077795A3 has a different approach, it is directed mainly to the gastrointestinal flora and to reduce or prevent diseases such as dysphagia and pneumonia: our invention, on the other hand, decreases and prevents, in a better proportion, different diseases derived from the oral microbiota, due to the effectiveness in the inoculation and state of our bacteria, since, thanks to the components used, our microorganisms can remain active, unlike the bacteria mentioned in the cited document, which only discloses the use of other microorganisms in an inactive manner.

Document WO2010077795A3 discloses that the compositions can have presentations in the form of tablets, lyophilized products, lollipops, sachets, soluble films, whereas our invention can be in liquid, semi-viscous and/or viscous form used as a filling of any confectionery product.

The present invention was developed as a result of the need to have an organoleptically friendly matrix for human consumption with natural components that give stability and viability to the active biomass of L. plantarum and S. salivarius integrated into the matrix, which when in contact with the mouth allows the reactivation and incorporation into the oral flora, inhibiting the bacterial biofilm adhered to the teeth, and reducing oral problems such as caries, bad breath, tartar, among others.

### OBJECTIVES OF THE INVENTION

The main objective of the present invention is to make available a matrix formulated with probiotics to provide stability to beneficial chemical-microbial properties in oral health that allows a decrease in the development of caries due to the inhibition of Streptococcus mutans and other pathogens that are the main causes of oral diseases.

Another objective of the invention is to make available said matrix formulated with probiotics to provide stability to beneficial chemical-microbial properties in oral health and that this matrix also offers viability and stability to the microorganisms Lactobacillus plantarum and Streptococcus salivarius which, upon contact with the mouth, manage to colonize and strengthen the oral flora.

Another objective of the invention is to provide said matrix formulated with probiotics to provide stability to beneficial chemical-microbial properties in oral health and that this matrix also allows microorganisms to be kept viable and stable in an active state that, upon contact with the mouth, manage to colonize the oral flora.

Another objective of the invention is to provide said matrix formulated with probiotics to provide stability to beneficial chemical-microbial properties in oral health and that this matrix also combats caries formation, prevents bad breath, and eliminates tartar and other oral conditions.

And all those qualities and objectives that will become apparent when carrying out a general and detailed description of the present invention based on the illustrated modalities.

### BRIEF DESCRIPTION OF THE INVENTION

In order to develop the matrix formulated with probiotics to provide stability to beneficial chemical-microbial properties in oral health, the process began with research and selection of the active component (microorganisms), performing a susceptibility test by means of filter paper discs onto which different probiotic microorganisms or natural substances were poured; then, they were left to dry (2-4 min) and placed in Petri dishes seeded with the caries consortium, including Streptococcus mutans. They were left incubating for 24 hours at 37° C. and checked every 12 h to determine inhibition. After 24 hours, an inhibition zone could be observed around the rings impregnated with Streptococcus salivarius and Lactobacillus plantarum, which surprisingly demonstrated the antimicrobial effect of these probiotics against the caries consortium.

Surprisingly, it was found that Streptococcus salivarius microorganisms manage to produce two classes of antibiotic bacteriocins: salivaricin A2 and salivaricin B, both strongly antagonistic to the growth of Streptococcus mutans, which in 95% of cases is the main cause of caries formation.

On the other hand, it was determined that Lactobacillus plantarum has greater hydrophobicity and adhesion affinity than Streptococcus mutans, since it contains the lac 4 and lac 5 genes that produce a greater amount of Beta-D-galactosidase, counteracting the cohesion of the pathogen. Tests were carried out in which the microorganisms Streptococcus salivarius and Lactobacillus plantarum were lyophilized in a tablet; said tablet was tested to determine the viability of the strain and its effectiveness since they are a type of dental probiotic.

To test its effect, the tablet was dissolved in BHI medium (infusion of calf brain and heart, peptic hydrolyzate from animal tissues, pancreatic hydrolyzate of casein, sodium chloride, glucose, disodium phosphate), it was left incubating for 24 hours and after that, it was examined under a microscope to determine the number of cells; however, no growth was obtained because in this state the microorganisms require an acid medium to reactivate, so it was concluded that said tablet only has an effect at the gastrointestinal level; consequently, a medium that simulates stomach acids (pH 4.5) was prepared.

The tablet was again dissolved in BHI medium (infusion of calf brain and heart, peptic hydrolyzate from animal tissues, pancreatic hydrolyzate of casein, sodium chloride, glucose, disodium phosphate) to which a medium simulating stomach acids (pH 4.5) was added, it was left incubating for 24 hours and after that, it was examined under a microscope where growth was observed. To validate that they were viable, they were seeded in a Petri dish and after 16 hours, colonies were obtained; when taking these colonies, they had a gummy consistency, which could be attributed to the compounds present in said tablets.

For a probiotic microorganism to have an effect in the human body, it has to be active and stable. Currently, the simplest method is the lyophilized presentation, this allows the microorganisms to be stable but inactive until activated by gastric juices. However, they do not interact with the oral flora but with the intestinal flora when they manage to colonize part of it. From here, it was determined that the active component (microorganisms) should be placed in a matrix that will allow its stability in an active and stable state to be effective.

To determine the composition of the matrix, several formulations were made focusing on four formulations, the first one consisted of a fruit-based rubbery paste which was inoculated with the microorganisms of interest, but there was no growth; the other three were carried out with a pectin base and only the flavoring was changed (cinnamon, cherry and mint).

Derived from them, it could surprisingly be found that the best composition of the matrix formulated to provide stability to beneficial chemical-microbial properties in oral health consists of a viscous solution, which is composed of pectin (3-5 g/100 mL), dextrose (6-9 g/100 mL), citric acid (25-50 mg/100 mL), and natural flavoring (2-4 mL/100 mL) selected from mint and spearmint; and as active ingredient, a mixture of microorganisms Streptococcus salivarius and Lactobacillus plantarum. When combined, these components form a protective barrier that allows microorganisms to be kept in stable conditions since pectin works as a natural thickener, which, when combined with sugar and citric acid, enables the formation of gels; these gels keep microorganisms stable while also exhibiting anti-adhesive activity against pathogens; on the other hand, dextrose provides the necessary nutrients to keep the microorganisms alive but without stimulating their proliferation; in turn, citric acid regulates the pH, increasing the conservation of the gel, and acts as an emulsifier; finally, the flavoring provides the organoleptic properties. The matrix provides viability and stability to the microorganisms Streptococcus salivarius and Lactobacillus plantarum which, upon contact with the mouth, manage to colonize and strengthen the oral flora.

The matrix of the present invention had an unexpected effect on the direct attack on the oral flora, since it reduces dental bacterial plaque, due to the active and stable presentation of probiotics within the matrix; this allows attacking and inhibiting Streptococcus mutans and other pathogens contained in the consortium, which are the main causes of diseases such as caries; that is, the greatest unexpected effect was the visible reduction of caries, both in the zones in Petri dishes, and in the extracted dental pieces that showed a high degree of contamination. In the case of dental pieces, the probiotic matrix was able to detach bacterial plaque and caries (black spots) non-invasively in just 5 days.

The formulated matrix as described herein can be used in confectionery, medicinal, and dental products, as well as in rinses, gums, etc.

The matrix provides stability and viability to the active ingredient (at least one microorganism in active state selected from Streptococcus salivarius and Lactobacillus plantarum or a mixture of both) that, upon contact with the mouth of a patient, allows easy colonization of the oral flora, inhibiting the bacterial biofilm adhered to the dental pieces, reducing oral problems such as caries, bad breath, tartar, among others; since these microorganisms stabilize the oral flora, counteracting the tropism generated by S. mutans.

Said at least one microorganism in active state selected from Streptococcus salivarius and Lactobacillus plantarum or a mixture of both in the formulated matrix, manage to inhibit S. mutans and the microbial consortium found in caries since the strains S. salivarius and L. plantarum produce antagonistic substances that inhibit the growth of S. mutans.

By nature, a classic or native biofilm forms on teeth, which is composed of bacteria embedded in a matrix of exopolysaccharides. Colonization is carried out from the growth of primitive bacteria (Actinomyces naeslundii and several species of streptococci) that are part of the oral microbiota, whose function is to strengthen oral health. Primitive bacteria, when adhering to dental surfaces, produce adhesins that facilitate the anchoring of secondary and tertiary bacteria. All these microorganisms are part of a microenvironment that under stable conditions provide well-being to the mouth. However, when there is an imbalance in the oral microbiota, the microorganisms form a bacterial plaque that appears to be a classic biofilm but causes the formation of caries that, if not treated, lead to tooth loss.

Therefore, the matrix developed in accordance with the present invention simulates the same mechanism since by keeping probiotic microorganisms viable, they manage to come in contact with bacterial plaque and inhibit the growth of species such as S. mutans; this allows to reduce the amount of pathogens, and at the same time the probiotic strains adhere through receptors or anchor proteins, allowing colonization with beneficial bacteria that help to rebalance the oral microbiota.

In the case of in vitro cultures (FIGS. 6a-8b) the reduction of pathogens is perceived in the reduction of black spots, which correspond to the formation of caries, just like in the in vivo setting.

When species such as Streptococcus salivarius and Lactobacillus plantarum, considered Generally Recognized As Safe (GRAS) by the Food and Drug Administration, commonly known by its English acronym FDA, are used as probiotic in the matrix formulated according to the present invention, they locally inhibit bacterial growth and adhesion, modulating the immune response through the production of antiviral agents.

The mechanism of action of probiotics is based on a nutritional competitiveness with oral microorganisms that are considered pathogens; with this, an ideal oral environment is created in which the risk of suffering from caries is reduced due to the production of antimicrobial substances such as bacteriocins and hydrogen peroxide.

The invention also contemplates a production process of the matrix formulated to provide stability to beneficial chemical-microbial properties in oral health, which comprises the following steps:
a) Pour distilled water into a beaker at a temperature between 80° C.-100° C., and add the ingredients in the following order: powdered dextrose [6-9 g/100 mL], powdered citric acid [25-50 mg/100 mL], powdered pectin [3-5 g/100 mL];
b) Shake at a temperature of 80° C. for 5-10 minutes at a speed of 250 to 500 rpm until dissolved;
c) Add flavoring[(1-2 mL/100 mL] and mix for 2-5 minutes at a speed of 100 to 150 rpm;
d) Pour the formulated matrix into a container, forming a maximum layer of 1 cm thick and sterilize it with UV light for 15 min, then pour it into a previously sterilized container for storage;
e) Prepare the inoculum by reactivating the strains of at least one microorganism selected from Streptococcus salivarius and Lactobacillus plantarum or a mixture of both, performing a count by means of a Neubauer chamber to determine the initial concentration, which will allow calculating the mL needed to obtain a final cell concentration of 4×10\hat{ }12 CFU/ml;
f) Take 1 mL aliquots and place them in Eppendorf tubes (the number of samples depends on the initial concentration) to perform three washes with physiological solution (2500 rpm, 5 min); after each wash, the microorganism pill or tablet is resuspended;
g) Finally, resuspend the tablet in sterile physiological solution (0.9% NaCl) and concentrate in the same diluent to obtain a final concentration of 4×10\hat{ }12 CFU/mL. This will ensure that for every 0.5 mL, there are 2×10\hat{ }9 CFU.
h) Inoculate the matrix by pouring 1.5-2 mL of the matrix into sterile Eppendorf tubes and inoculate each tube with the corresponding probiotic microorganism, injecting the cell suspension, mixing gently and storing at room temperature (25° C.) for 5 days;
i) The entire process from the preparation of the inoculum to the inoculation of the matrix is carried out in a laminar flow hood under aseptic conditions and with sterile material.

In order to better understand the characteristics of the invention, the present description is accompanied, as an integral part thereof, by the drawings with an illustrative but non-limiting nature, which are described below.

### BRIEF DESCRIPTION OF THE FIGURES

FIGS. 1a and 1b show a top view of a Petri dish where a disk with four radial divisions is defined with BHI medium (infusion of calf brain and heart, peptic hydrolyzate from animal tissues, pancreatic hydrolyzate of casein, sodium chloride, glucose, disodium phosphate) inoculated with a caries consortium and then inoculated with natural coconut and the microorganisms Saccharomyces Boulardii, Streptococcus salivarius and Lactobacillus plantarum.

FIGS. 2a and 2b show a top view of a Petri dish where a disc is defined with BHI medium (infusion of calf brain and heart, peptic hydrolyzate from animal tissues, pancreatic hydrolyzate of casein, sodium chloride, glucose, disodium phosphate) inoculated firstly with a caries consortium and then inoculated with Streptococcus salivarius and Lactobacillus plantarum, showing growth inhibition zones of the caries consortium in the areas where they were subsequently inoculated with Streptococcus salivarius and Lactobacillus plantarum.

FIG. 3 shows a top view of a Petri dish where a disk with nine radial divisions is defined with BHI culture medium (infusion of calf brain and heart, peptic hydrolyzate from animal tissues, pancreatic hydrolyzate of casein, sodium chloride, glucose, disodium phosphate), inoculated with Streptococcus salivarius to determine the viability of the strains contained in the matrices with cinnamon, mint and cherry flavorings, defining three quadrants (each division corresponds to 11% of the total area of the dish).

FIGS. 4a and 4b show a top view of two Petri dishes where a disc is defined with six radial divisions (each division corresponds to 16% of the total area of the dish) with BHI culture medium (infusion of calf brain and heart, hydrolyzed peptide from animal tissues, pancreatic hydrolyzate of casein, sodium chloride, glucose, disodium phosphate), one was inoculated with Streptococcus salivarius and the other with Lactobacillus plantarum to determine the viability of the strains contained in the matrices.

FIG. 4c shows a top view of four Petri dishes where a disc with six radial divisions is defined in each one (each division corresponds to 16% of the total area of the dish) with BHI culture medium (infusion of calf brain and heart, peptic hydrolyzate from animal tissues, pancreatic hydrolyzate of casein, sodium chloride, glucose, disodium phosphate), one was inoculated with Streptococcus salivarius and the other with Lactobacillus plantarum to determine the viability of the strains contained in twelve matrices; among the twelve matrices prepared, six maintained the viability of the strains, and this is evident in the formation of colonies in FIGS. 4a and 4b.

FIG. 5 shows a top view of five Petri dishes that were first seeded with a caries consortium and subsequently, the rings containing the probiotic matrix with different flavorings were placed on them. For the two dishes on the left, the matrix included the microorganism Streptococcus salivarius and for the two dishes on the right, the matrix included the microorganism Lactobacillus plantarum, while the central dish served as a matrix control dish with water.

FIGS. 6a and 6b show the before and after of a decayed tooth with a caries in a support, which has been treated with the mint flavoring matrix of the present invention in an in vitro test.

FIGS. 7a and 7b show the before and after of other decayed tooth with a caries in a support, which has been treated with the mint flavoring matrix of the present invention in an in vitro test.

FIGS. 8a and 8b show the before and after of other decayed tooth with a caries in a support, which has been treated with the spearmint flavoring matrix of the present invention in an in vitro test.

FIGS. 9a and 9b show the before and after of other decayed tooth with a caries in a support, to which the matrix without the prebiotic microorganisms has been applied in a control in vitro test.

FIGS. 10a and 10b show a before and after photograph of the oral cavity of a patient exhibiting the dentition, who was treated with the matrix formulated in accordance with the present invention incorporated into chewing gums, which were chewed twice a day for one week.

For a better understanding of the invention, a detailed description of some of its modalities will be made, shown in the drawings that are attached to this description with an illustrative but non-limiting nature.

### DETAILED DESCRIPTION OF THE INVENTION

The characteristic details of the matrix formulated to provide stability to beneficial chemical-microbial properties in oral health, in accordance with the present invention, are clearly shown in the following detailed description and in the attached illustrative drawings, serving the same reference signs to indicate the same parts.

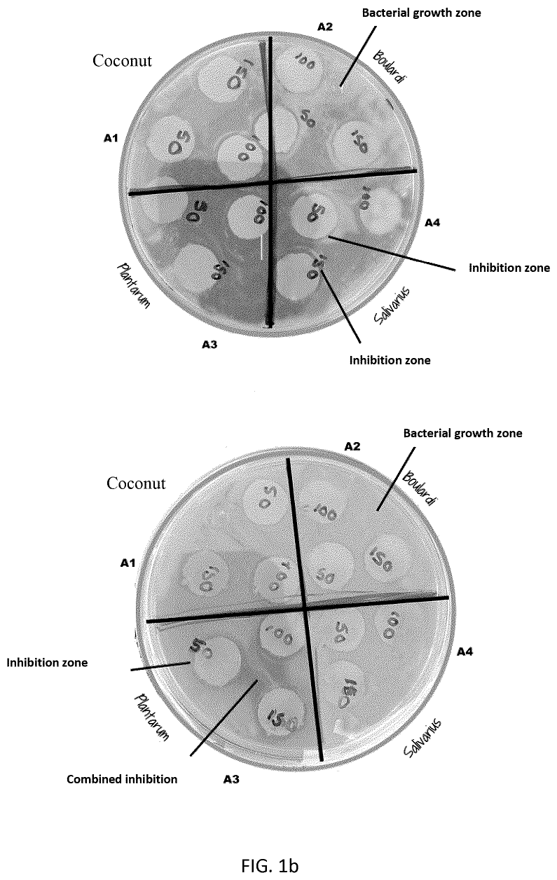

According to FIGS. 1a and 1b, two dishes were prepared with BHI medium (infusion of calf brain and heart, peptic hydrolyzate from animal tissues, pancreatic hydrolyzate of casein, sodium chloride, glucose, disodium phosphate), which were first inoculated with a caries consortium; after that, filter paper discs with 50, 100 y 150 μL were inoculated with natural substances such as natural coconut and the microorganisms Saccharomyces Boulardii, Streptococcus salivarius and Lactobacillus plantarum. They were allowed to dry and were placed with the help of a sterile forceps on the surface of the dishes. The Petri dishes were left incubating at 37° C. for 24 hours.

The objective of this experiment was to determine the components and inhibitory amounts of the caries consortium made up mainly of Streptococcus mutans.

TABLE 1
Components for the test with inhibition rings

| Radial | Inhibitor | % of |
|---|---|---|
| division | component | division |
| A-1 | Natural coconut | 25 |
| A-2 | S. boulardii | 25 |
| A-3 | S. salivarius | 25 |
| A-4 | L. plantarum | 25 |
| B-1 | Natural coconut | 25 |
| B-2 | S. boulardii | 25 |
| B-3 | S. salivarius | 25 |
| B-4 | L. plantarum | 25 |

The above was an experiment using the Kirby-Bauer technique, and the following results were obtained. The discs impregnated with Streptococcus salivarius and Lactobacillus plantarum (A-3, A-IV, B-III and B-IV) showed a high interference activity against microorganisms of the caries consortium due to a combined inhibition. While the other components did not show any sign of inhibition.

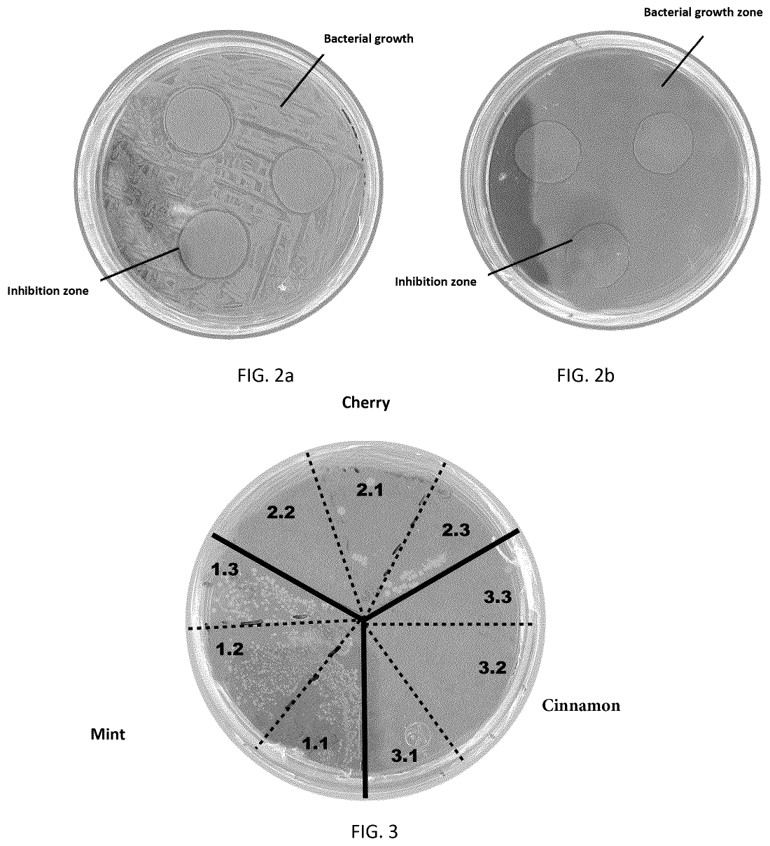

According to FIGS. 2a and 2b, derived from the experiment in accordance with Table 1 and FIGS. 1a and 1b, it was decided to carry out the same experiment, but only with these two microorganisms (Streptococcus salivarius and Lactobacillus plantarum), and as shown in said FIGS. 2a and 2b, inhibition zones of approximately 3±0.3 cm in diameter can be observed in each of the rings; in said inhibition zone, there is no bacterial growth of the strain originating from caries, this indicates that indeed the 100 μL of the solution containing both probiotic strains had an inhibitory effect on the growth of the bacterial consortium.

To determine the composition of the matrix, several formulations were carried out, finding that those that included pectin, dextrose and a natural flavoring (cinnamon, cherry and mint) offered the best results in stability for the probiotic microorganisms Streptococcus salivarius and Lactobacillus plantarum, as shown in table 2 and FIG. 3.

TABLE 2
Design of experiments to determine the concentration of the different reagents

| | | Pectin | | Citric | | | |
|---|---|---|---|---|---|---|---|
| Radial | Favoring | [g/20 | Dextrose | acid[mg/20 | | Active | % of |
| division | [ml/20 ml] | ml] | [g/20 ml] | ml] | Flavoring | microorganism | division |
| 1.1 | 0.5 | 0.75 | 1.5 | 3 | Mint | S. salivarius | 11% |
| 1.2 | 1 | 1.25 | 2.25 | 5 | Mint | S. salivarius | 11% |
| 1.3 | 2 | 1.75 | 3 | 9 | Mint | S. salivarius | 11% |
| 2.1 | 0.5 | 0.75 | 1.5 | 3 | Cherry | S. salivarius | 11% |
| 2.2 | 1 | 1.25 | 2.25 | 5 | Cherry | S. salivarius | 11% |
| 2.3 | 2 | 1.75 | 3 | 9 | Cherry | S. salivarius | 11% |
| 3.1 | 0.5 | 0.75 | 1.5 | 3 | Cinnamon | S. salivarius | 11% |
| 3.2 | 1 | 1.25 | 2.25 | 5 | Cinnamon | S. salivarius | 11% |
| 3.3 | 2 | 1.75 | 3 | 9 | Cinnamon | S. salivarius | 11% |

According to FIG. 3, a Petri dish with BHI medium (infusion of calf brain and heart, peptic hydrolyzate of animal tissues, pancreatic hydrolyzate of casein, sodium chloride, glucose, disodium phosphate) is shown, divided radially, where each space was seeded with each matrix prepared based on Table 2. This test was carried out to determine the ideal concentration of each ingredient based on the growth of the probiotic microorganisms contained in them, since this would indicate their stability and active presence of the strains. In turn, several flavorings were evaluated in order to select the one that would not interfere with the growth and viability of probiotic microorganisms.

FIG. 3 shows a top view of a Petri dish where a disk with nine radial divisions (each division corresponds to 11% of the total area of the dish) is defined with BHI culture medium (infusion of calf brain and heart, peptic hydrolyzate from animal tissues, pancreatic hydrolyzate of casein, sodium chloride, glucose, disodium phosphate), inoculated with Streptococcus salivarius to determine the viability of the strains contained in the matrices with cinnamon, mint and cherry flavorings, defining three quadrants 1.1, 1.2, 1.3 for mint; 2.1, 2.2, 2.3 for cherry and 3.1, 3.2, 3.3 for cinnamon. It can be observed that the quadrants that did not show inhibition were the formulations with the Mint flavoring (quadrants 1.1, 1.2, 1.3), concluding that the cherry and cinnamon flavorings have antimicrobial components. The first one contains various polyphenolic agents, while the second one contains between 70-95% eugenol, components used as bactericides.

Based on the results obtained in the first design of experiments, a second design was carried out (Table 3) where the same concentrations of all the reagents were tested again, but this time the two probiotic strains Streptococcus salivarius and Lactobacillus plantarum were included; spearmint was added as a new flavoring, it has similar characteristics to mint, and three brands of both flavorings were evaluated to determine those that did not inhibit the growth of microorganisms.

TABLE 3
Design of experiments to determine the concentration of the different reagents.

| Radial | Flavoring | Pectin | Dextrose | Citric acid | | Active | % of |
|---|---|---|---|---|---|---|---|
| division | [ml/20 ml] | [g/20 ml] | [g/20 ml] | [mg/20 ml] | Flavoring | microorganism | division |
| 1 | 1.5 | 1.75 | 3 | 7 | Spearmint | S. salivarius/ | 16% |
| | | | | | | L. plantarum | |
| 2 | 1.5 | 1.75 | 3 | 7 | Spearmint | S. salivarius/ | 16% |
| | | | | | | L. plantarum | |
| 3 | 1.5 | 1.75 | 3 | 7 | Mint | S. salivarius/ | 16% |
| | | | | | | L. plantarum | |
| 4 | 1.5 | 1.75 | 3 | 7 | Mint | S. salivarius/ | 16% |
| | | | | | | L. plantarum | |
| 5 | 0.5 | 0.75 | 1.5 | 3 | Mint | S. salivarius/ | 16% |
| | | | | | | L. plantarum | |
| 6 | 0.5 | 0.75 | 1.5 | 3 | Mint | S. salivarius/ | 16% |
| | | | | | | L. plantarum | |
| 7 | 0.5 | 0.75 | 1.5 | 3 | Spearmint | S. salivarius/ | 16% |
| | | | | | | L. plantarum | |
| 8 | 0.5 | 0.75 | 1.5 | 3 | Spearmint | S. salivarius/ | 16% |
| | | | | | | L. plantarum | |
| 9 | 1 | 1.25 | 2.25 | 5 | Spearmint | S. salivarius/ | 16% |
| | | | | | | L. plantarum | |
| 10 | 1 | 1.25 | 2.25 | 5 | Mint | S. salivarius/ | 16% |
| | | | | | | L. plantarum | |
| 11 | 1 | 1.25 | 2.25 | 5 | Mint | S. salivarius/ | 16% |
| | | | | | | L. plantarum | |
| 12 | 1 | 1.25 | 2.25 | 5 | Spearmint | S. salivarius/ | 16% |
| | | | | | | L. plantarum | |

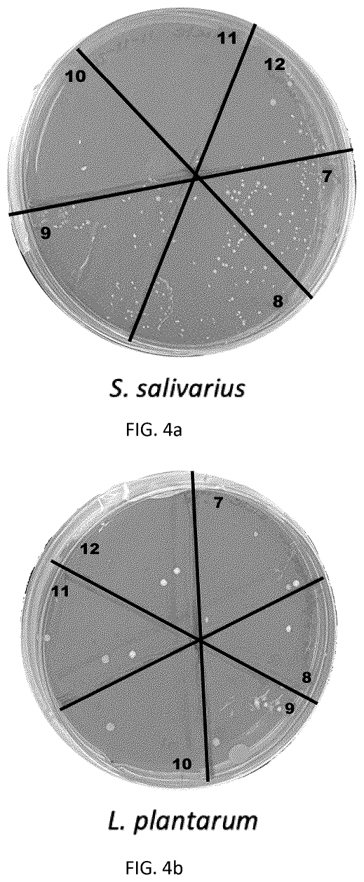

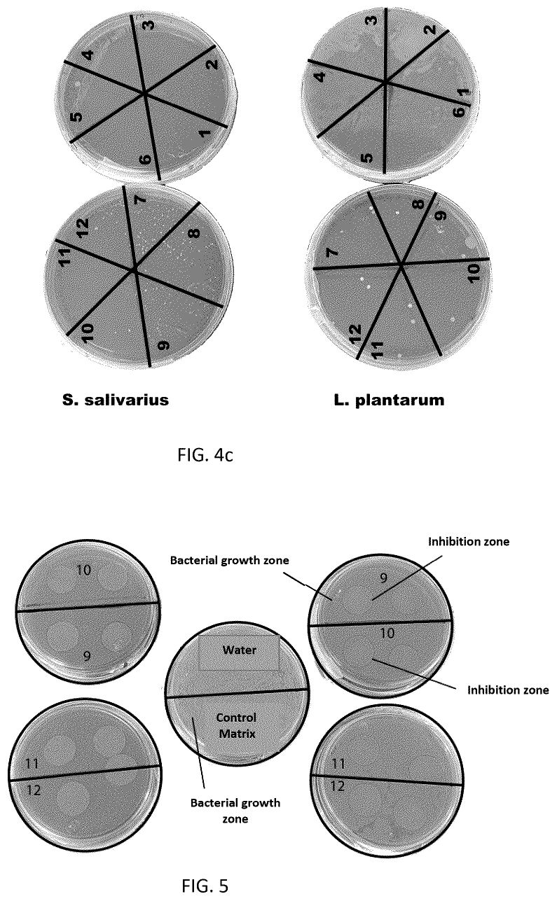

As seen in FIG. 4c, six of the twelve prepared matrices maintained the viability of the strains, which is reflected in colony formation shown in FIGS. 4a and 4b. With this result, it was verified that the average concentration is adequate to keep the strains alive and viable within the matrix at room temperature, since the properties of each component work together to promote the development of the microorganisms used.

With the results obtained, it was possible to establish a range for each component of the matrix.
a) Pectin (3-5 g/100 ml),
b) Dextrose (6-9 g/100 ml),
c) Citric acid (25-50 mg/100 ml), and
d) Natural flavoring-Mint/Spearmint (1-2 mL/100 ml),
e) Microorganisms S. salivarius and/or
f) Microorganisms L. plantarum

TABLE 4
Properties of the matrix at 27 ± 2° X.

| Physical Properties | Chemical properties |
|---|---|
| Color: | pH: 4 ± 2 |
| Semitransparent | |
| Odor: Mint or | Degrees Brix: 13° |
| spearmint | |
| Flavor: Mint or | Viscosity: 1.56 ± 0.3 CPS |
| spearmint | |

To determine the appropriate flavorings, a susceptibility test was performed with each of the matrices, in which sterile filter paper rings were impregnated with 100 μL of each flavoring and left incubating for 24 hours.

According to FIG. 5, five Petri dishes are shown, each Petri dish was first seeded with a caries consortium and subsequently, the rings containing the probiotic matrix with different flavorings were placed on them. After the incubation time, the dishes with matrices 9-12 presented an inhibition zone of approximately 1-2 mm around each one of the rings, which indicates that the matrix together with the probiotics manages to inhibit the growth of the microorganisms from caries; this result was corroborated by evaluating that the rings of the control dish (water and matrix with flavoring without probiotic) did not present any inhibition zone.

To determine the concentration of the matrix components, an experiment design was carried out (Tables 2 and 3), by means of which there was an evaluation of the stability of the matrix through the growth (conservation) of the microorganisms, its viability (FIGS. 4a and 4b) and subsequently its inhibitory effect on the consortium of bacteria from caries (FIG. 5).

For the flavoring selection process, the viability of the strains contained in each of the matrices was determined; to do that, a 10 μL sample was taken, then seeded in BHI medium (infusion of calf brain and heart, peptic hydrolyzate from animal tissues, pancreatic hydrolyzate of casein, sodium chloride, glucose, disodium phosphate) and allowed to incubate for 24 hours.

It was concluded that the matrix should be formed with the aforementioned amounts, since the microorganisms have the necessary nutrients to keep them in the stationary phase; in addition, under these amounts, the matrix can be out of refrigeration without suffering any alteration. To verify the viability of the strains, a seeded was performed after 30 days, which showed a stationary state behavior with the desired minimal growth.

In the case of microorganisms, they are removed from the BHI medium (infusion of calf brain and heart, peptic hydrolyzate from animal tissues, pancreatic hydrolyzate of casein, sodium chloride, glucose, disodium phosphate) when they reach the stationary phase, they are subjected to three washes with physiological solution to prevent cells from being lysed by osmotic pressure and then the pellet is resuspended and concentrated in sterile physiological solution (0.9% NaCl) to reach the final concentration of 4×10\hat{ }12 CFU/mL. This will ensure that for every 0.5 mL, there are 2×10\hat{ }9 CFU.

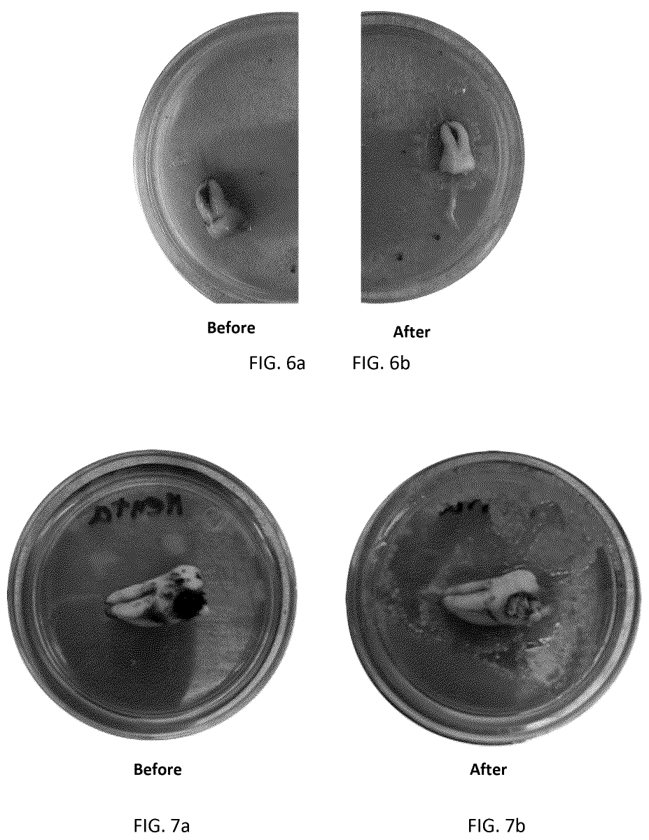

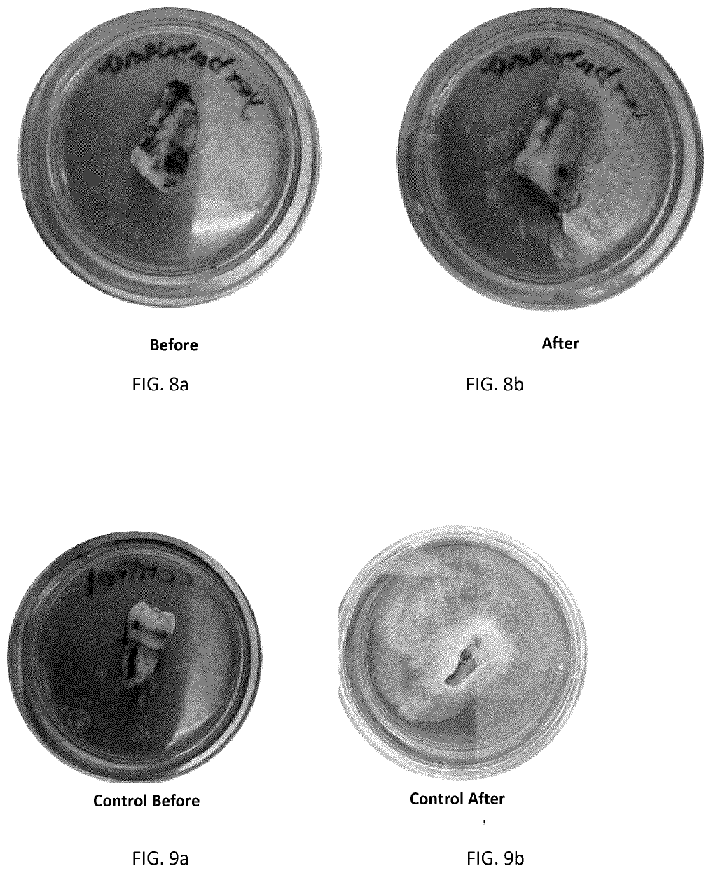

According to FIGS. 6a-9b, to determine the functioning of the matrix, dental pieces affected by caries were selected, placed in BHI medium (infusion of calf brain and heart, peptic hydrolyzate from animal tissues, pancreatic hydrolyzate of casein, sodium chloride, glucose, disodium phosphate) and with the help of a sterile syringe, the pieces were covered with 100 μL of the corresponding matrix, every two times a day for 5 days and between each application, the remains of the previous matrix were removed with a sterile swab. After each application the Petri dishes were stored in the incubator at 37° C. to simulate mouth temperature.

With the in vitro study, it was possible to determine that the matrix by itself (without the probiotic microorganisms Streptococcus salivarius and Lactobacillus plantarum), does not have a positive response since after 2 days, the control dental piece was contaminated, which caused the caries to increase, darkening the tooth in almost its entirety. In cases where the matrix formulated in accordance with the present invention was applied, it inhibited the bacterial biofilm adhered to the teeth, reducing the caries problem; therefore, it can eliminate bad breath, tartar, among other conditions derived from the tropism generated by S. mutans.

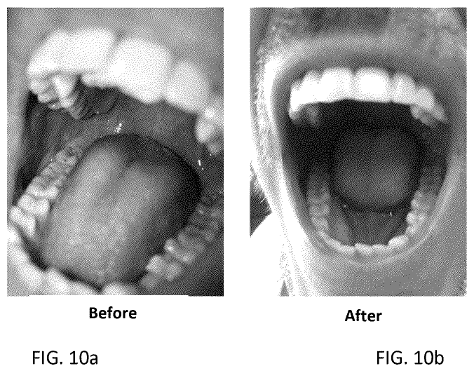

This was done qualitatively, and for this purpose, five individuals with some type of caries were selected, and they were given the product to consume twice a day for one week. A photograph of a patient was taken at the beginning of the tests and another at the end of the tests to observe the decrease in caries (see FIGS. 10a and 10b), in addition, they were asked if they had felt any changes when consuming the gum with the probiotic matrix, and all of them reported feeling a reduction in bacterial plaque upon first contact with chewing gums containing the formulated matrix of the present invention.

The invention has been sufficiently described so that a person with average knowledge in the matter can reproduce and obtain the results that we mention in the present invention. However, any person skilled in the field of the technique corresponding to the present invention may be able to make modifications not described in the present application; however, if the application of such modifications in a specific structure or manufacturing process thereof requires the subject matter claimed in the following claims, said structures should be understood within the scope of the invention.

1. A matrix formulated with probiotics to provide stability to beneficial chemical-microbial properties in oral health, characterized by comprising pectin, dextrose, citric acid, natural flavoring and an active ingredient consisting of at least one microorganism selected from Streptococcus salivarius and Lactobacillus plantarum or a mixture of both.

2. The matrix formulated with probiotics to provide stability to beneficial chemical-microbial properties in oral health according to claim 1, characterized by comprising 3-5 g/100 mL of pectin, 6-9 g/100 mL of dextrose, 25-50 mg/100 mL of citric acid, 2-4 mL/100 mL of natural flavoring; and 4×10\hat{ }12 CFU/mL of a microorganism selected from Streptococcus salivarius and Lactobacillus plantarum or a mixture of both.

3. The matrix formulated with probiotics to provide stability to beneficial chemical-microbial properties in oral health according claim 1, wherein the natural flavoring is selected from mint and spearmint.

4. The matrix formulated with probiotics to provide stability to beneficial chemical-microbial properties in oral health according to claim 1, wherein the probiotic is a viscous solution.

5. The matrix formulated with probiotics to provide stability to beneficial chemical-microbial properties in oral health according to claim 1, wherein the pectin acts as a natural thickener that, when combined with sugar and citric acid, allows the formation of gels, which keep microorganisms stable and at the same time develop an anti-adhesive activity against pathogens; where dextrose provides necessary nutrients to microorganisms to keep them alive, but without stimulating their proliferation; where said citric acid regulates the pH, increasing the conservation of the gel and acts as an emulsifier, and where the flavoring provides the organoleptic properties.

6. The matrix formulated with probiotics to provide stability to beneficial chemical-microbial properties in oral health according to claim 1, wherein the at least one microorganism selected from Streptococcus salivarius and Lactobacillus plantarum or a mixture of both, are in active state.

7. The matrix formulated with probiotics to provide stability to beneficial chemical-microbial properties in oral health according to claim 1, wherein the probiotics are integrated into confectionery, medicinal, dental, rinse and gum products.

8. A production process of the matrix formulated with probiotics to provide stability to beneficial chemical-microbial properties in oral health with a composition of claim 1, comprises the following steps:
a) pour distilled water into a beaker at a temperature between 80° C.-100° C., and add the ingredients in the following order: powdered dextrose [6-9 g/100 mL], powdered citric acid [25-50 mg/100 mL], powdered pectin [3-5 g/100 mL];
b) shake at a temperature of 80° C. for 5-10 minutes at a speed of 250 to 500 rpm until dissolved;
c) add flavoring[(1-2 mL/100 mL] and mix for 2-5 minutes at a speed of 100 to 150 rpm;
d) pour the formulated matrix into a container, forming a maximum layer of 1 cm thick and sterilize it with UV light for 15 min, then pour it into a previously sterilized container for storage;
e) prepare the inoculum by reactivating the strains of at least one microorganism selected from Streptococcus salivarius and Lactobacillus plantarum or a mixture of both, performing a count by means of a Neubauer chamber to determine the initial concentration, which will allow calculating the mL needed to obtain a final cell concentration of 4×10\hat{ }12 UFC/mL;
f) take 1 mL aliquots and place them in Eppendorf tubes (the number of samples depends on the initial concentration) to perform three washes with physiological solution (2500 rpm, 5 min); after each wash, the microorganism pill or tablet is resuspended;
g) finally, resuspend the tablet in sterile physiological solution (0.9% NaCl) and concentrate in the same diluent to obtain a final concentration of 4×10\hat{ }12 UFC/mL; and
h) inoculate the matrix by pouring 1.5-2 mL of the matrix into sterile Eppendorf tubes and inoculate each tube with the corresponding probiotic microorganism, injecting the cell suspension, mixing gently and storing at room temperature (25° C.) for 5 days;

9. The production process of the matrix formulated with probiotics according to claim 8, wherein stages “e” to “h” are carried out in a laminar flow hood under aseptic conditions and with sterile material.

10. The use of a matrix formulated with probiotics to improve oral health comprising the fact of administering a patient a composition of claim 1.

11. The use of a matrix formulated with according to claim 10, wherein the probiotics counteracts or prevents caries, prevents bad breath, removes tartar, removes yellowing from teeth and other oral conditions.
